(19) United States
(12) Patent Application Publication
(10) Pub. No.: US 2024/0215878 A1
(43) Pub. Date: Jul. 4, 2024

(54) BLOOD COLLECTION ASSEMBLY AND METHOD OF USING THE SAME

(71) Applicant: MICRO LAB SOLUTIONS, LLC, Jupiter, FL (US)

(72) Inventor: Ryan EL-HOSSEINY, Jupiter, FL (US)

(21) Appl. No.: 18/564,706

(22) PCT Filed: Aug. 10, 2021
(86) PCT No.: PCT/US2021/071151

§ 371 Date: Nov. 28, 2023

Related U.S. Application Data
(60) Provisional application No. 63/180,449, filed on Apr. 27, 2021.

(51) Int. Cl. A61B 5/15; B01L 9/06

(52) U.S. Cl. CPC A61B 5/150267; A61B 5/150076; A61B 5/150259; A61B 5/150343; A61B 5/150351; B01L 9/06

(57) ABSTRACT

A blood collection tube funnel, comprising a funnel having a spout, a stand fixedly secured to the funnel and the spout, and a base secured to a distal end of the stand. The stand farther includes a transverse support member having a gripping means arranged to removably secure a blood collection tube thereon. A method of using the blood collection tube funnel is also provided.

### CROSS-REFERENCE TO RELATED APPLICATIONS

This application claims priority to U.S. Provisional Patent Application No. 63/180,449, filed Apr. 27, 2021, which application is incorporated herein by reference in its entirety.

### FIELD OF THE INVENTION

The present invention relates generally to a blood collection assembly, specifically to a blood collection assembly having a gripping mechanism arranged to secure a blood collection tube thereon.

### REARGROUND OF THE INVENTION

Blood collection tubes are well known in the art and are common medical devices. These tubes are used in a variety of medical procedures and particularly for diagnostic purposes where a blood sample is needed to test various chemical and substance levels to assess the health of a patient. In many of these cases, a filled blood collection tube is sent to a lab where a centrifuge will separate the blood into the plasma, buffy coat (white blood cells and platelets), and red blood cells. Typically, a patient would employ a medical provider to conduct the blood collection.

Inversely, at-home blood collection has become more commonplace where smaller amounts of blood will satisfy a given diagnostic procedure. In the home setting, typically a patient receives a package including a blood collection tube to fill and send off for testing. In this at-home blood collection setting, it can be difficult for patients to properly fill a blood collection tube to a desirable and testable level due to a smaller size of the at-home collection tubes.

Thus, there is a long felt need for a device that facilitates filling a smaller-diameter blood collection tube that includes a configuration to securely hold the tube during a blood-filling process.

### SUMMARY OF THE INVENTION

The present invention broadly comprises a blood collection assembly including a funnel having a spout, a stand fixedly secured to the funnel and the spout, and a base secured to a distal end of the stand, wherein the stand comprises a pair of support legs.

The present invention further comprises at least one transverse support member fixedly secured to the pair of support legs, the pair of support legs each comprise a top end and a bottom end, the top end of each of the pair of support legs includes a support fin fixedly secured thereon, the support fin is further arranged to fixedly secure to a sloped outer section of the funnel. The at least one transverse support member also includes a gripping means. The gripping means is arranged to frictionally secure a blood collection tube thereto.

The present invention also comprises a blood collection assembly, comprising a base, a stand fixedly secured to the base, the stand comprising a pair of support legs, at least one transverse support member fixedly secured to each of the pair of support legs, a funnel having a spout, the funnel fixedly secured to the stand, and, a cap arranged to removably engage the spout, wherein the at least transverse support member includes a gripping mechanism arranged to removably secure a blood collection tube therein.

A primary object of the invention is to provide a device that facilitates filling a smaller-diameter blood collection tube that includes a configuration to securely hold the tube during a blood-filling process.

Another object of the invention is to provide a method for using the blood collection assembly comprising the steps of removing a warm-up pad and the blood collection assembly from a sealed container, heating the warm-up pad, placing a blood collection tube underneath a distal end of the spout, placing a finger into the warm-up pad for a period of time and applying pressure to the warm-up pad during the period of time, removing the finger from the warm-up pad and sterilizing the finger, lancing the finger to draw blood, and, aiming the finger over the funnel of the blood collection assembly to drop blood into the funnel to collect blood in the blood collection tube.

These and other objects, features and advantages of the invention will become readily apparent to one having ordinary skill in the art upon study of the following detailed description in view of the drawings and appended claims.

### BRIEF DESCRIPTION OF THE DRAWINGS

Various embodiments are disclosed, by way of example only, with reference to the accompanying schematic drawings in which corresponding reference symbols indicate corresponding parts, in which:

FIG. 1 is a perspective view of the first embodiment of the present invention;

FIG. 2 is a front elevation view of the first embodiment of the present invention shown in FIG. 1;

FIG. 3 is a rear elevation view of the first embodiment of the present invention shown in FIG. 1;

FIG. 4 is a left side view of the first embodiment of the present invention shown in FIG. 1;

FIG. 5 is a top plan view of the first and second embodiments of the present invention;

FIG. 6 is a bottom view of the first and second embodiments of the present invention;

FIG. 7 is a perspective view of the first embodiment of the present invention shown in FIG. 1 with the cap engaged;

FIG. 8 is a perspective view of the second embodiment of the present invention;

FIG. 9 is a front elevation view of the second embodiment of the present invention shown in FIG. 8;

FIG. 10 is a perspective view of the second embodiment of the present invention shown in FIG. 8 with the cap engaged;

FIG. 11 is a front elevation view of the first embodiment of the present invention shown in FIG. 1 with a blood collection tube engaged to the gripping mechanism and the spout;

FIG. 12 is a front elevation view of the first embodiment of the present invention shown in FIG. 1 with a blood collection tube engaged to the gripping mechanism and the base aperture;

FIG. 13A is a partial perspective view of the invention, illustrating alternative gripping means 70 and clamp 72 of the present invention;

FIG. 13B is a partial perspective view of the invention, illustrating alternative gripping means 80 and sleeve 82 of the present invention;

FIG. 13C is a partial perspective view of the invention, illustrating gripping means 61 having protrusions 63; and,

FIG. 13D is a partial perspective view of the invention, illustrating gripping means 92 which is affixed to only leg 32.

### DETAILED DESCRIPTION OF THE PREFERRED EMBODIMENT

At the outset, it should be appreciated that like reference numbers on different drawing figures represent identical structural elements. It should also be appreciated that, while a number of different embodiments and variations of the present invention are shown in the various drawings, the invention as claimed is not intended to be limited to these specific embodiments, as the claims define a broader invention that can take many different shapes and structures. Also, the adjectives, “top”, “bottom”, “right”, “left”, and their derivatives, in the description herebelow, refer to the perspective of one facing the invention as shown in the figure under discussion.

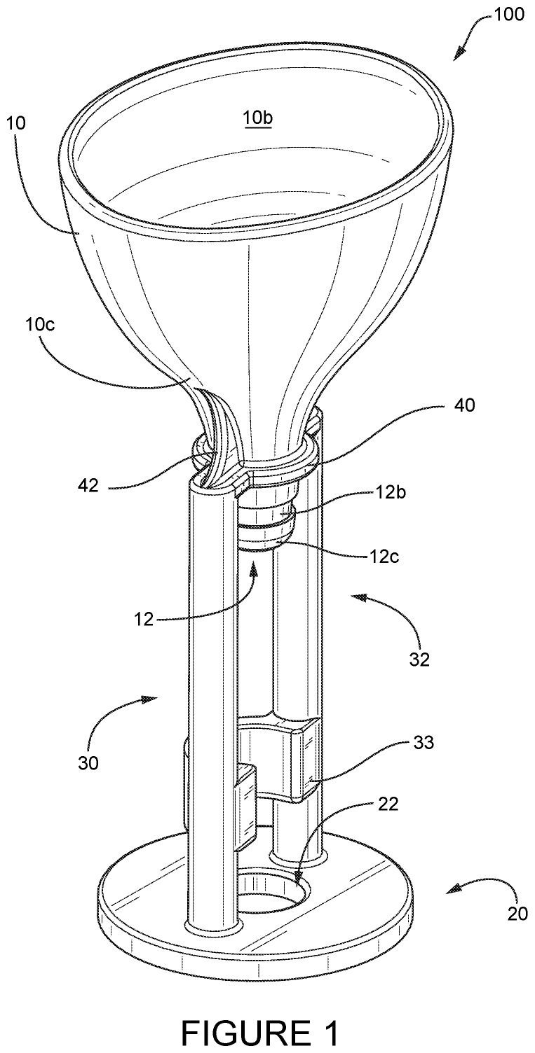

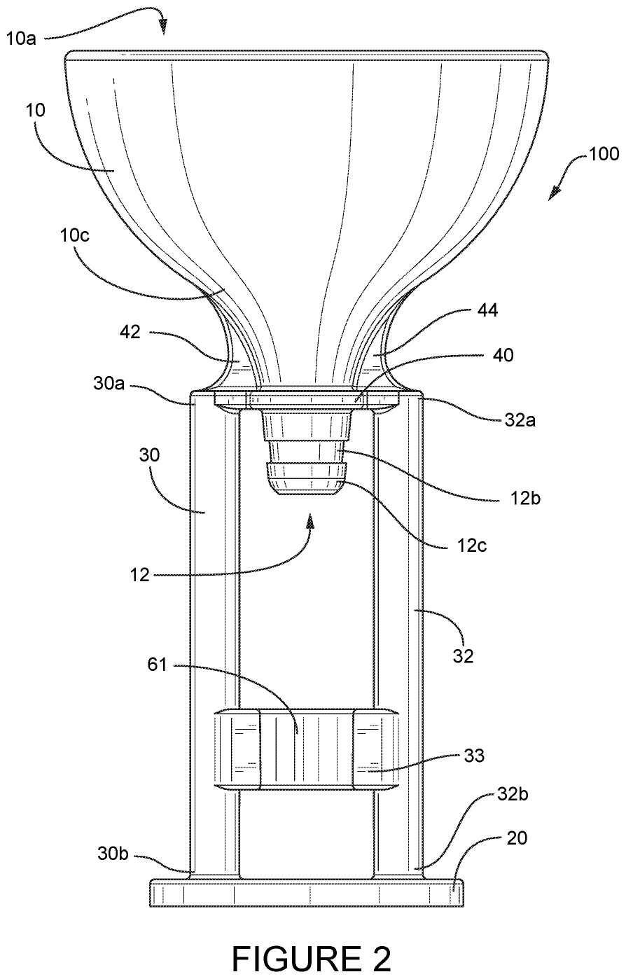

Adverting now to the figures. FIG. 1 illustrates the first embodiment of the blood collection assembly. Broadly, blood collection assembly 100 comprises base 20, a stand (comprising legs 30 and 32), and funnel 10. In a preferred embodiment, base 20 is annular in shape and comprises aperture 22. The base could obviously be constructed in other shapes. Extending vertically from a top surface of base 20 is the stand. In a preferred embodiment, the stand comprises two support legs, first support leg 30 and second support leg 32. First leg 30 and second leg 32 are preferably fixedly secured to the top surface of base 20 at their respective distal ends. At the opposite ends of first leg 30 and second leg 32 is support ring 40, first support fin 42 and second support fin 44 (shown in FIG. 2). Support ring 40 is fixedly and perpendicularly secured to an inside surface of both first leg 30 and second leg 32. First support fin 42 and second support fin 44 are fixedly secured to the top surface of first leg 30 and second leg 32, respectively. Support ring 40 circumscribes outer sloped surface 10c of funnel 10. First support fin 42 and second support fin 44 extend upwardly from first leg 30 and second leg 32 and are fixedly secured on opposite sides of outer sloped surface 10c of funnel 10. Support ring 40, first support fin 42 and second support fin 44 collectively secure funnel 10 to the stand.

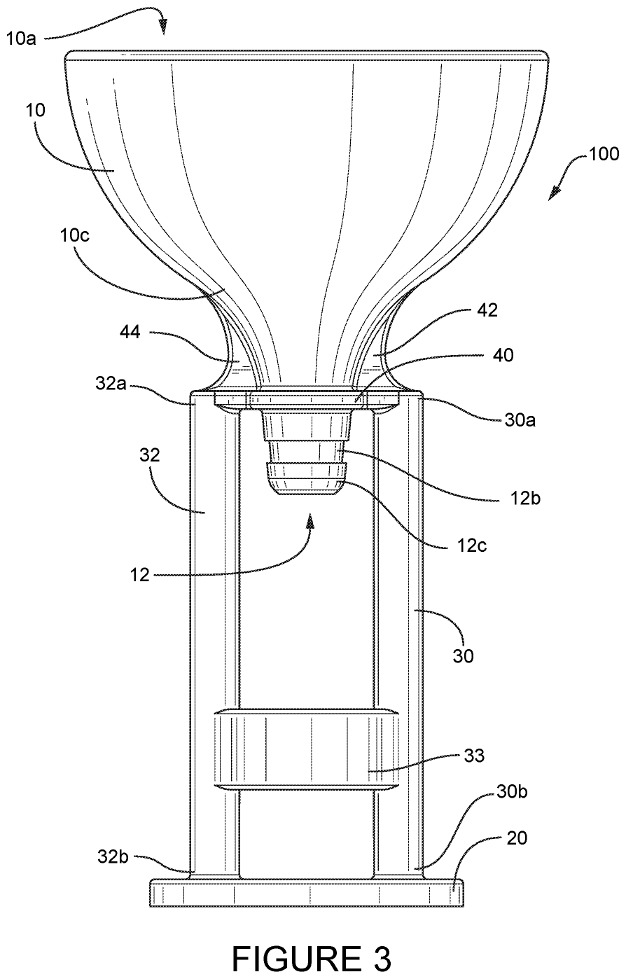

The following description should be taken in view of FIGS. 1 through 3. FIG. 2 illustrates a front elevation view of blood collection assembly 100 and FIG. 3 illustrates a rear elevation view of blood collection assembly 100. Funnel 10 comprises opening 10a, inside surface 10b, outer sloped surface 10c, and spout 12. Funnel 10 tapers from a larger inner and outer circumference at its top end to a smaller inner and outer diameter at its distal bottom end. Outer sloped surface 10c terminates into spout 12 below support ring 40. Spout 12 comprises tip 12c and groove 12b. In a preferred embodiment, the shape of the funnel resembles that of an inverted bathroom plunger. It should be appreciated, however, that the funnel could be made in a myriad of different shapes, including frustoconical, conical, elliptical or a combination of semi-spherical/hyperbolic shapes, although, ideally, the funnel maintains a larger diameter opening at its top than at its bottom. In a preferred embodiment, the funnel has a wide opening at the top, sloping sides, and a narrower opening or tube at its bottom, configured to pour and guide liquid into a container, such as a tube, located beneath its bottom opening.

First support leg 30 includes top end 30a and distal end 30b. Distal end 30b of first support leg 30 is fixedly secured to the top surface of base 20. First support fin 42 and support ring 40 are fixedly secured to top end 30a of first support leg 30. Second support leg 32 includes top end 32a and distal end 32b. Distal end 32b of second support leg 32 is fixedly secured to the top surface of base 20. Second support fin 44 and support ring 40 are both fixedly secured to top end 30b of second support leg 32. Transverse support member 33 is fixedly secured to first support leg 30 and second support leg 32. Transverse support member 33 is preferably biased in a position that is closer to distal end 30b of first support leg 30 and distal end 32b of second support leg 32. Transverse support member 33 includes gripping means 61, described in further detail infra.

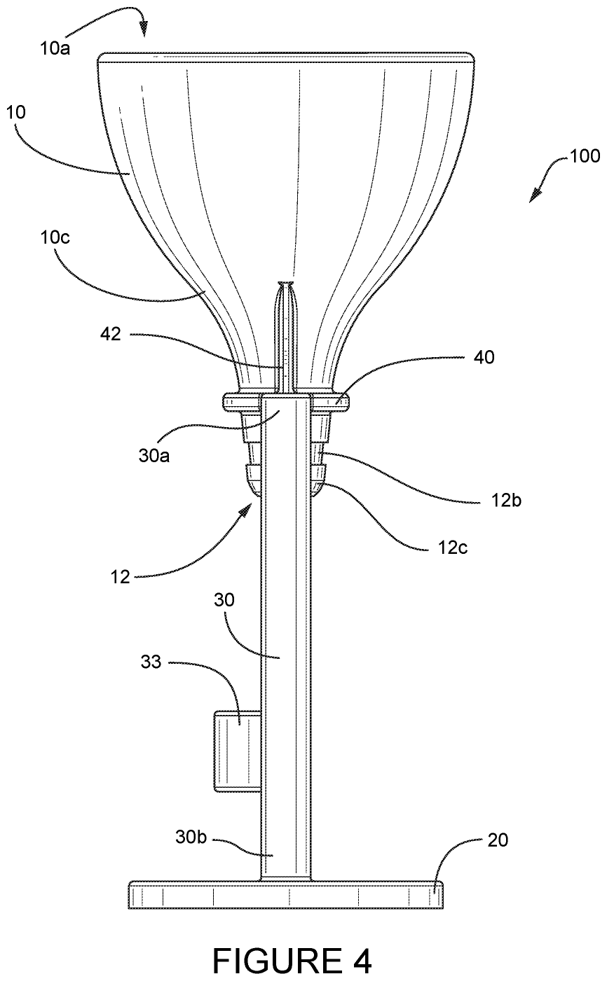

The following description should be taken in view of the preceding Figures and FIG. 4. FIG. 4 illustrates a left side view of blood collection assembly 100. Transverse support member 33 extends inwardly from both first support leg 30 and second support leg 32 before curving in a direction away from both first support leg 30 and second support leg 32. The curve is shown in the figures to extend to the rear of blood collection assembly 100. However, it should be appreciated that the “front” or “rear” is interchangeable based on the view of blood collection assembly 100. The curved feature of transverse support member 33 is gripping means 61. Specifically, gripping means 61 is curved in a manner that allows the outer surface of gripping means 61 to frictionally secure to an outer surface of a blood collection assembly, securing it thereto.

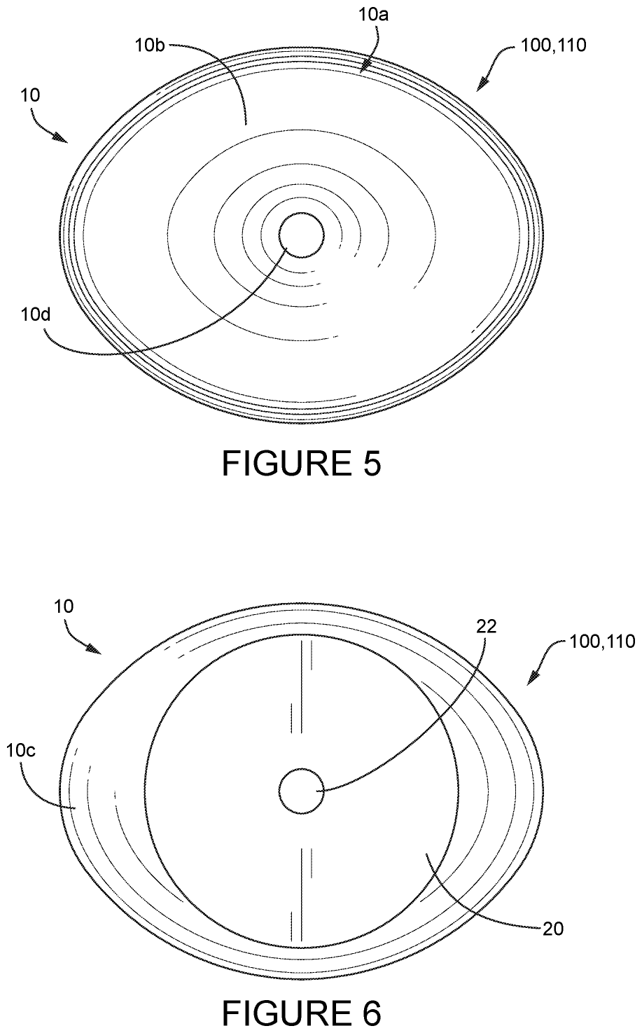

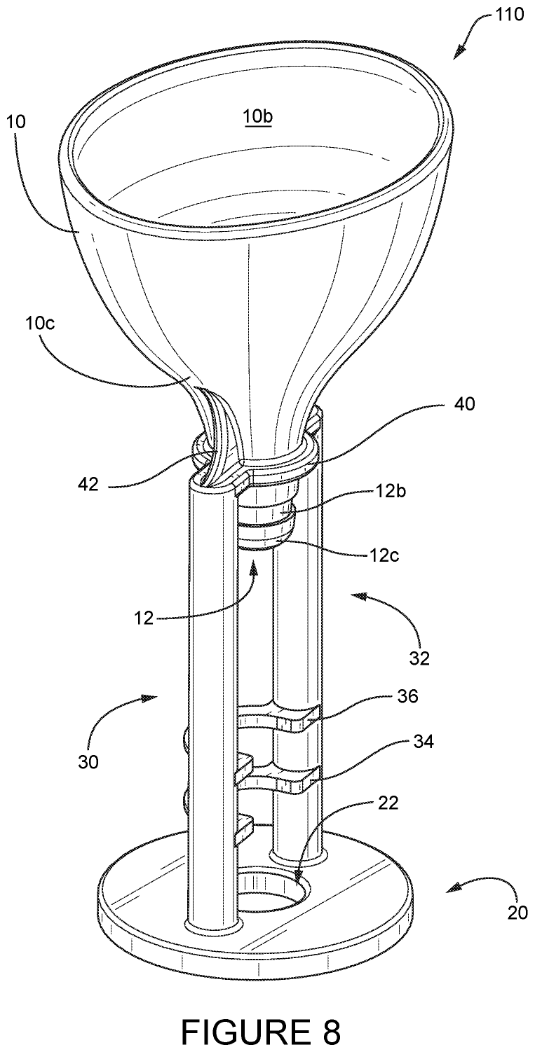

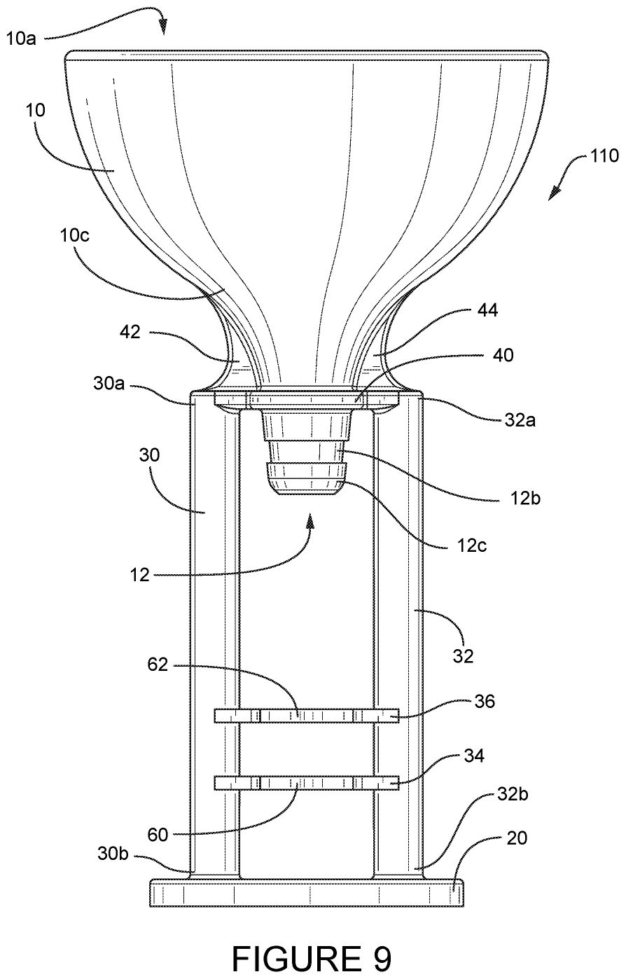

The following description should be taken in view of FIGS. 5 and 6. FIG. 5 illustrates a top view of blood collection assembly 100 and FIG. 6 illustrates a bottom view of blood collection assembly 100. It should be appreciated that FIGS. 5 and 6 also illustrate the top and bottom views of a second embodiment of the present invention, blood collection assembly 110 which is described infra in view of FIGS. 8-9. In a preferred embodiment, funnel 10 has a generally elliptically-shaped base and an apex. The base is opening 10a of funnel 10 (described and shown supra) and the apex is spout 12. Inside surface 10b of funnel 10 slopes until aperture 10d of funnel 10. Aperture 10d is continuous and merges into spout 12 of funnel 10. Inside surface 10b and sloped outside surface 10c have a decreasing circumference as inside surface 10b and sloped outside surface 10c get closer to aperture 10d and spout 12, respectively. FIG. 6 illustrates base 20 and aperture 22 of base 20. It should be appreciated that aperture 22 of base 20 has a center point that is colinear with aperture 10d of funnel 10. Aperture 22 of base 20 is preferably circular. Aperture 22 has an inner circumference/diameter that is slightly larger or approximately equal to an outside circumference/diameter of a blood collection tube, allowing the blood collection tube to be frictionally secured within the funnel and yet easily removable therefrom. Aperture 22 of base 20 may have a different inner circumference depending on the size of an outer perimeter of a blood collection tube selected for use, as the primary purpose of aperture 22 is to provide for secondary securement of the blood collection tube in addition to at least one gripping means.

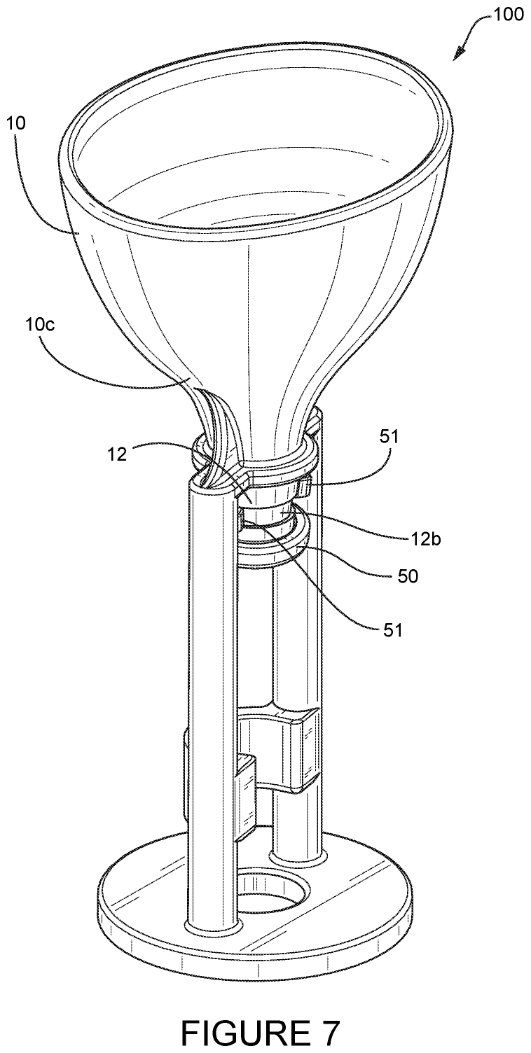

FIG. 7 illustrates a perspective view of blood collection assembly 100 with cap 50 secured to spout 12 of funnel 10. Cap 50 includes locking means 51, which, in the preferred embodiment, is a semi-circular clasp that extends upwardly from a base of cap 50. Locking means 51 of cap 50 is arranged to partially surround and engage groove 12b of spout 12 and frictionally secure sloped end 12c of spout 12 within a partial through-bore within the base of cap 50. Although the embodiment of cap 50 and spout 12 illustrates a snap-fit type locking mechanism of locking means 51, it should be appreciated that both locking means 51 of cap 50 and sloped end 12c of spout 12 may alternatively be threaded to lock cap 50 securely on spout 12. A threaded locking connection may be a preferable alternative in circumstances where blood collection assembly 100 may be used with in conjunction with diagnostic materials being added to a blood collection tube, or in circumstances where blood residue in the funnel needs to be prevented from dripping from the spout after collection.

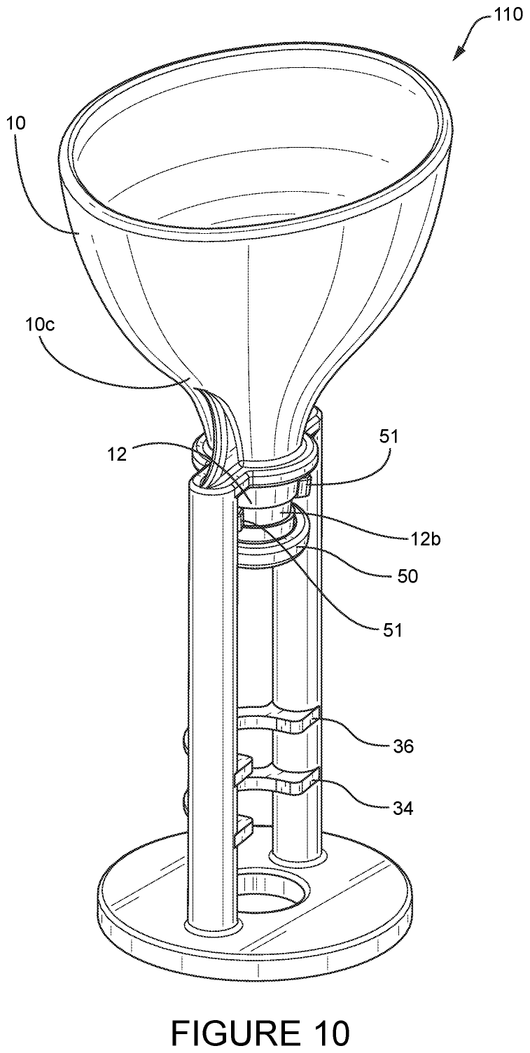

FIGS. 8 through 10 illustrate an alternative embodiment of the present invention. Blood collection assembly 110 includes all of the features described supra in view of the preferred embodiment of present invention, blood collection assembly 100, except for a pair of transverse support members instead of a single transverse support member. First transverse support member 34 and second transverse support member 36 are both fixedly secured to first support leg 30 and second support leg 32. First transverse support member 34 and second transverse support member 36 are both located in between top end 30a and distal end 30b of first support leg 30 and between top end 32a and distal end 32b of second support leg and are preferably located in a position that is closer to the distal ends 30b, 32b of first and second support legs 30, 32. First transverse support member 34 includes gripping means 60 and second transverse support member 36 includes gripping means 62. Gripping means 60 and 62 are structurally and functionally the same as described supra in view of gripping means 61 of transverse support member 31 except that they are individually smaller in height compared to said transverse support member 31.

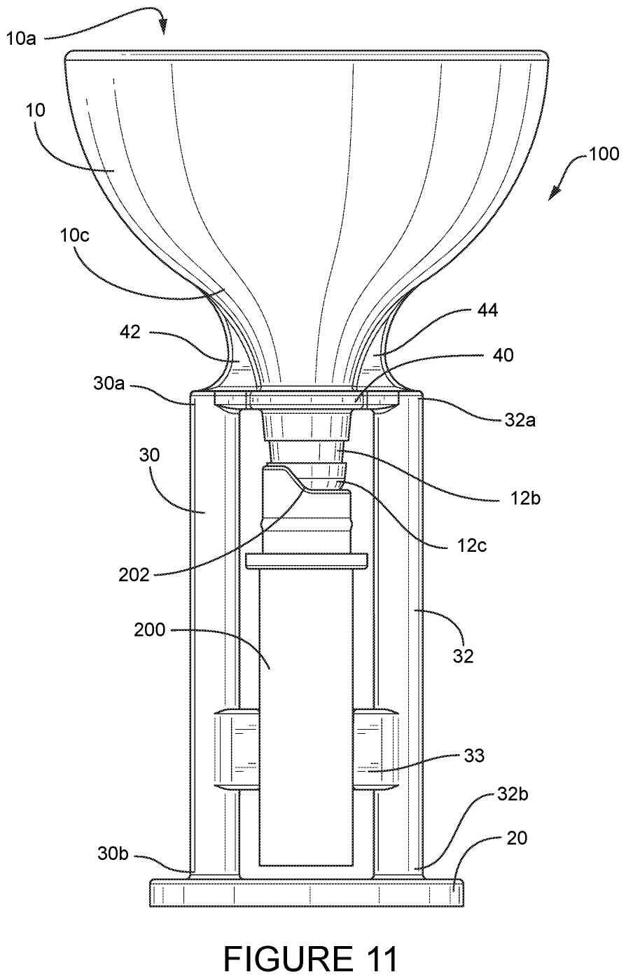

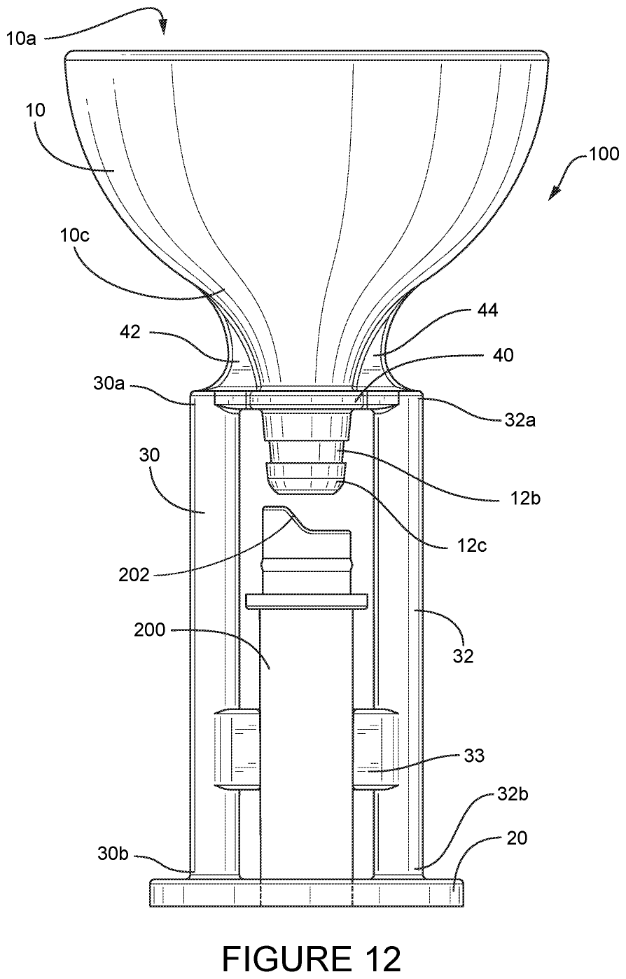

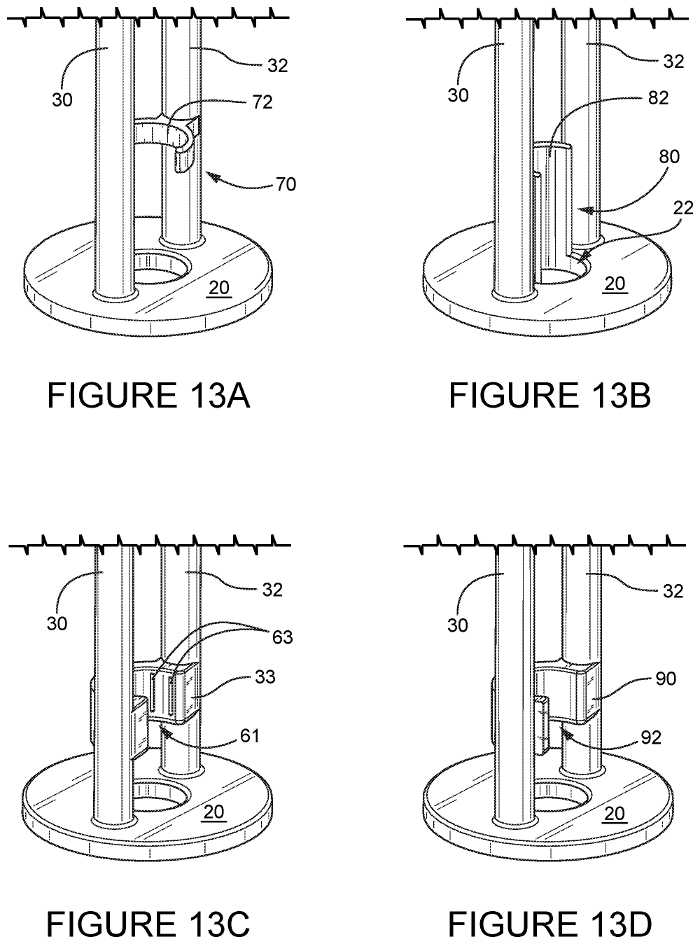

FIGS. 11 and 12 illustrate blood collection tube 200 engaged to blood collection assembly 100. It should be appreciated that the following description should be taken in view of all of exemplary figures and applies to both the preferred embodiment of the present invention (blood collection assembly 100) and to the secondary or alternative embodiment of the present invention (blood collection assembly 110), along with the alternative gripping means as shown in FIGS. 13A through 13D. Gripping means 61 of transverse support member 31 is arranged to frictionally secure an outer surface of blood collection tube 200. Gripping means 61 is preferably a semi-circular shape and have an inner circumference that is approximately identical to the outer circumference of blood collection tube 200 to allow tube 200 to be pressed into gripping means 61 to engage a frictional securement therein. Gripping means 61 may have different inner circumferences in relation to the outer circumference of a desired blood collection tube. Blood collection tube 200 includes opening 202. Sloped end 12c of spout 12 is arranged to have an outer circumference that is slightly smaller than the inner circumference of opening 202 of blood collection tube 200 allowing a section of sloped end 12c to be frictionally and securely inserted within opening 202 of blood collection tube 200. It should be appreciated that alternative sized gripping means 61 accommodates larger or smaller blood collection tubes and it should also be appreciated that the outer circumference of sloped end 12c of spout 12 changes accordingly. Aperture 22 of base 20 (shown in FIGS. 1, 6, and 8) is also sized in relation to the outer circumference of blood collection tube 200 such that an inner circumference of aperture 22 is approximately the same or slightly larger than the outer circumference of blood collection tube 200 to afford a fictional grip. For example, if blood collection tube 200 is manually slid in a downward direction within gripping means 61, as shown in FIG. 12, a space is created between opening 202 and sloped end 12c so cap 50 may be engaged to spout 12 to prevent blood spillage after collection. The inner circumference of gripping means 61; the outer circumference of sloped end 12c of spout 12; and, the inner circumference of aperture 22 of base 20 all are sized based on the outer circumference of blood collection tube 200 and the inner circumference of opening 202 of blood collection tube. It should also be appreciated that the inner circumference of gripping means 61; the outer circumference of sloped end 12c of spout 12; and, the inner circumference of aperture 22 of base 20 all have a center point which are collinearly arranged.

FIG. 13A illustrates a partial perspective view of base 20, stand (support legs 30 and 32), and an alternative embodiment of the gripping means. Alternative gripping means 70 is preferably clamp 72 and is arranged to have an inner circumference that is approximately the same as the outer circumference of a blood collection tube, such that clamp 72 of alternative gripping means 70 will frictionally secure a blood collection tube therein. Gripping means 70 is fixedly secured to either first support leg 30 or second support leg 32.

FIG. 13B illustrates a partial perspective view of base 20, stand (support legs 30 and 32), and a further alternative embodiment of the gripping means. Alternative gripping means 80 is preferably sleeve 82 and is arranged to have an inner circumference that is approximately the same as the outer circumference of a blood collection tube, such that sleeve 82 of gripping means 80 will frictionally secure a blood collection tube therein. Gripping means 80 is a semi-circular extension of aperture 22 of base 20.

FIG. 13C illustrates a partial perspective view of base 20, stand (support legs 30 and 32), and gripping means 61 of transverse member 33. Gripping means 61 includes plurality of protrusions 63 that are arranged on the inside curve of gripping means 61. Plurality of protrusions 63 may be added to any of the previously described gripping means and provide further frictional securement of a blood collection tube that is inserted within any of the embodiments of the gripping means.

FIG. 13D illustrates a partial perspective view of base 20, stand (support legs 30 and 32), and a still further alternative embodiment of the gripping means. Alternative transverse member 90 is fixedly secured to either first support leg 30 or second support leg 32 and includes gripping means 90. Gripping means 90 is essentially the same as gripping means 33 except that transverse member 90, which gripping means 90 is integral with, does not secure to both support legs.

It should be appreciated that gripping means 61, 60 and 62, 72, 82, and/or protrusions 63, may be interchangeable within blood collection assembly 100. In view of the preceding description and corresponding figures, the gripping means may be affixed to the stand on one, or both of the support legs, or alternatively may be an extension of aperture 22 of base 20, as shown in FIG. 13B via sleeve 82.

The following description should be taken in view of all of the previous described figures. The size of opening 10a of funnel 10 is significantly greater than the opening of blood collection tube 200. This difference in size, allows blood to drip from a patient's finger or foot anywhere into opening 10a, where the blood droplets will collect and fall into aperture 10d of funnel 10, to then pass-through spout 12, out through sloped end 12c of spout 12, and into blood collection tube 200. Opening 10a of funnel 10 is significantly larger than the opening at the distal end of sloped end 12c of spout 12. Without blood collection assembly 100, 110, a patient may have increased difficultly in aiming a blood droplet coming from the bottom of their finger, into a small diameter opening of blood collection tube 200 or from a foot of a child in pediatric setting where infants are unable to keep their bodies still. Blood collection assembly 100, 110 ensures that all blood that drips from a patient's finger or foot is collected and passed into blood collection tube 200, providing for a more efficient and easier blood sample collection process in an at-home or clinical setting. It should also be appreciated that, although opening 10a of funnel 10 is shown to have a generally oval-shape configuration, different shaped opening may be used to accommodate different patients and settings.

It should also be appreciated that although the figures illustrate the funnel of the present invention fixedly secured to the stand of the present invention, the funnel may be removable, such that it is a snap-fit or threadably connectable to the stand to allow for collapsibility, storage, or in medical laboratory settings where small volumes of materials may be added to the blood collected in the blood collection tube via a micropipette.

The following description should be taken in view of the previously described figures and details a preferred method of using blood collection assembly 100, 110. To increase the effectiveness of blood collection in conjunction with blood collection assembly 100, 110 the following steps are preferred to provide the quickest and cleanest method of providing a sample to be collected by blood collection assembly 100 and blood collection tube 200.

Step 1: Removing a warm-up pad, blood collection assembly 100 or 110, blood collection tube 200, and a lancet from a sealed container;

Step 2: Heating the warm-up pad. (In a preferred embodiment the pad may be heated in a microwave oven at a high setting for approximately 45 seconds. It should be appreciated that other methods may be used to heat the pad such as, but not limited to immersion in a water bath, or heating the pad via induction, radiation, or convection methods. It should also be appreciated that different patient's heat tolerances may require heating in a microwave oven for fewer than 45 seconds (e.g., 30 seconds), and patients with poor circulation may need to heat the warm-up pad for longer than 45 seconds);

Step 3: Removing a cap or seal from opening 202 of blood collection tube 200 and inserting blood collection tube 200 into gripping means 61 (or any of the other gripping means embodiments discussed supra);

Step 4: Sliding blood collection tube 200 upwardly to engage opening 202 of blood collection tube 200 onto sloped end 12c of spout 12;

Step 5: Placing a finger into the warm-up pad for approximately 30 seconds and applying pressure to the warm-up pad during the 30 seconds. Pressure may be applied to the finger within the warm-up pad by using a patient's opposite hand or a weight. However, it should be appreciated that the period of time a patient's finger is within the warm-up pad and the period of time pressure is applied to the finger may vary depending on the quality of blood circulation of the patient and/or the amount of blood that is needed for the collection; Step 6: Removing the patient's finger from the warm-up pad and sterilizing the patient's finger. For example, a patient may sterilize their finger with a disposable alcohol pad;

Step 7: Lancing the patient's finger with the lancet to draw blood;

Step 8: Aiming the patient's finger over opening 10a of funnel 10 to drop blood into funnel 10 to collect blood in blood collection tube 200;

Step 9: Sliding blood collection tube 200 in a downward direction within gripping means 61 (or any of the other gripping means embodiments discussed supra) to disengage opening 202 of blood collection tube 200 with sloped end 12c of spout 12; and,

Step 10: Engaging cap 50 with spout 12 to prevent blood spillage.

Thus, it is seen that the objects of the invention are efficiently obtained, although modifications and changes to the invention may be readily imagined by those having ordinary skill in the art, and these changes and modifications are intended to be within the scope of the claims.

### REFERENCE NUMBERS

10 Funnel
10a Opening of Funnel 10
10b Inside Surface of Funnel 10
10c Sloped Outer Surface of Funnel 10
10d Aperture of Funnel 10 and Spout 12
12 Spout of Funnel 10
12b Groove of Spout 12
12c Sloped End of Spout 12
20 Annular Base
22 Tube Aperture of Annular Base 20
30 First Support Leg
30a Top End of First Support Leg 30
30b Distal End of First Support Leg 30
32 Second Support Leg
32a Top End of Second Support Leg 32
32b Distal End of Second Support Leg 32
33 Transverse Support Member
34 First Transverse Support Member
36 Second Transverse Support Member
40 Support Ring
42 First Joining Support Fin
44 Second Joining Support Fin
50 Cap
51 Locking Means of Cap 50
60 Gripping Means of First Transverse Support Member 34
61 Gripping Means of Transverse Support Member 33
62 Gripping Means of Second Transverse Support Member 36
63 Protrusion of Gripping Means 61
70 Alternative Gripping Means
72 Clamp of Alternative Gripping Means 70
80 Alternative Gripping Means
82 Sleeve of Alternative Gripping Means 80
90 Alternative Transverse Member
92 Gripping Means of Alternative Transverse Member 90
100 Blood Collection Assembly
110 Blood Collection Assembly
200 Blood Collection Tube
202 Opening of Blood Collection Tube 200

1. A blood collection assembly, comprising:
a base;
a stand fixedly secured to said base, said stand comprises a pair of support legs;
a funnel having a spout, said funnel fixedly secured to said stand;
at least one transverse support member fixedly secured to said pair of support legs; and,
at least one gripping means arranged on said at least one transverse support member, wherein said gripping means is arranged to frictionally secure a blood collection tube therein.

2-4. (canceled)

5. The blood collection assembly recited in claim 31, wherein said pair of support legs each comprises a top end and a bottom end, said top end of each of said pair of support legs includes a support fin fixedly secured thereon, said support fin is further arranged to fixedly secure to a sloped outer section of said funnel.

6. The blood collection assembly recited in claim 1, wherein said spout is arranged distally in relation to an opening of said funnel.

7. The blood collection assembly recited in claim 1 further comprising:
a cap having a locking means arranged to secure to said spout.

8. The blood collection assembly recited in claim 1, wherein said base comprises an aperture, said aperture of said base is further arranged to frictionally secure a blood collection tube therein.

9. The blood collection assembly recited in claim 41, wherein said funnel comprises a support ring, said support ring is arranged on an outer surface of said funnel between said sloped section of said funnel and said spout, said support ring is fixedly secured to a top end of each of said pair of support legs.

10. The blood collection assembly recited in claim 7, wherein said spout further includes a groove circumscribing an outer surface of said spout, said groove is further arranged to mate with said locking means of said cap.

11. The blood collection assembly recited in claim 10, wherein said cap comprises a partial through-bore, said partial through-bore is further arranged to accept a distal surface of said spout when engaged thereto.

12. The blood collection assembly recited in claim 1, wherein said spout is further arranged to have a sloped outer surface, said sloped outer surface is further arranged to fixedly secure to an inside surface of an opening of a blood collection tube frictionally securing said blood collection tube to said spout.

13. The blood collection assembly recited in claim 6, wherein said opening of said funnel is an elliptical shape.

14. The blood collection assembly recited in claim 1, wherein said funnel is removable from said stand.

15. The blood collection assembly recited in claim 31, wherein said stand further comprises a pair of transverse support members fixedly secured to said pair of support legs.

16. The blood collection assembly recited in claim 15, wherein said pair of transverse support members each include a gripping means.

17. A method for using the blood collection assembly recited in claim 1, comprising the steps of:
removing a warm-up pad and said blood collection assembly from a sealed container;
heating said warm-up;
inserting an opening of a blood collection tube onto a distal end of said spout;
placing a finger into said warm-up pad for a period of time and applying pressure to said warm-up pad during said period of time;
removing said finger from said warm-up pad and sterilizing said finger;
lancing said finger to draw blood; and,
aiming said finger over said funnel to drop blood into said funnel of said blood collection assembly to collect blood in said blood collection tube.

18. A blood collection assembly, comprising:
a base;
a stand fixedly secured to said base, said stand comprising a pair of support legs;
at least one transverse support member fixedly secured to each of said pair of support legs;
a funnel having a spout, said funnel fixedly secured to said stand; and,
a cap arranged to removably engage said spout,
wherein each of said at least one transverse support member includes a gripping means arranged to removably secure a blood collection tube therein.

19. The blood collection assembly recited in claim 18, wherein said spout has a center point and an opening of said blood collection tube has a center point, wherein said gripping means are further arranged to position said center point of said opening of said blood collection tube engaged thereon collinearly with said center point of said spout.

20. A method for using the blood collection assembly recited in claim 19, comprising the steps of:
removing a warm-up pad and said blood collection assembly from a sealed container;
heating said warm-up pad;
inserting said blood collection tube into said gripping means;
sliding blood collection tube upwardly to engage said opening of said blood collection tube onto a distal end of said spout;
placing a finger into said warm-up pad for a period of time and applying pressure to said warm-up pad during said period of time;
removing said finger from said warm-up pad and sterilizing said finger;
lancing said finger to draw blood;
aiming said finger over said funnel to drop blood into said funnel of said blood collection assembly to collect blood in said blood collection tube;
sliding said blood collection tube in a downward direction within said gripping means to disengage said opening of said blood collection tube with said distal end of said spout; and,
engaging said cap with said spout.
